(19) United States
(12) Patent Application Publication
(10) Pub. No.: US 2014/0135919 A1
(43) Pub. Date: May 15, 2014

(54) FREE-FORM PROGRESSIVE MULTIFOCAL REFRACTIVE LENS FOR CATARACT AND REFRACTIVE SURGERY

(71) Applicant: STAAR Surgical Company, Monrovia, CA (US)

(72) Inventors: Ivair Gontijo, Los Angeles, CA (US); Thomas R. Paul, Westlake Village, CA (US); Alexei Ossipov, Laguna Niguel, CA (US)

(73) Assignee: STAAR Surgical Company, Monrovia, CA (US)

(21) Appl. No.: 14/076,097

(22) Filed: Nov. 8, 2013

Related U.S. Application Data
(60) Provisional application No. 61/724,842, filed on Nov. 9, 2012.

(51) Int. Cl. G02C 7/06; A61F 2/16

(52) U.S. Cl. CPC G02C 7/061; A61F 2/1637
USPC 623/6.24; 351/159.42

(57) ABSTRACT

A new type of multi-focal lens that has a free-form progressive multifocal front surface consisting of a 16th order polynomial superimposed on a standard conic base surface is described. The center region of the lens is optimized for distance vision, while simultaneously optimizing the rest of the lens for near vision. The resulting free-form even asphere polynomial surface is smooth, unlike present day diffractive multifocal designs. Additionally, this lens design is suitable for both refractive and cataract surgeries.

### CROSS-REFERENCES TO RELATED APPLICATIONS

This application claims priority from U.S. Application No. 61/724,842, filed Nov. 9, 2012, incorporated by reference in its entirety.

### BACKGROUND

After the onset of presbyopia the crystalline lens in the human eye can no longer accommodate to allow focusing on objects at a distance and nearby objects such as books or computer screens. The simplest solution to this problem consists of wearing spectacles for distance vision and reading glasses for near vision. The next step in sophistication to solve this problem is the use of bifocal lenses in spectacles so that the patient can look straight ahead through a lens for distance vision or “look down” through a lens of different power (but part of the same piece of glass on the frame) for near vision.

Two other solutions have been implemented that are more sophisticated. First there are so called pseudo-accommodation lenses that are implanted in the eye and are supposed to mimic the effect of the crystalline. The results and patient outcomes have been mixed at best. Although the FDA approved one of these lenses (Crystallens), many doctors and patients had poor experience with it and it has gone out of favor.

The other approach that has gained popularity consists of multifocal diffractive (MFD) lenses. It is very important to emphasize that these lenses are CATARACT lenses, i.e., they are implanted in patients of older age (normally 60 years old or above) that have developed cataract. Therefore, MFD lenses are primarily implanted to correct cataract problems and involve the extraction of the natural human crystalline lens and its replacement by the MFD lens. As an added bonus, MFD lenses are designed to restore a certain level of near vision, but this is not true accommodation. Such lenses do not accommodate, rather, they are designed to provide best focus for distance vision at the center of the lens and some degree of near vision at the periphery of the lens.

The simplest of MFD cataract lenses is the Restor® lens, made by Alcon. This lens has a center portion 3 mm in diameter designed for distance vision. Beyond this center portion there is a section sculpted with rings that changes the lens focal power, similar to Fresnel lenses invented over 150 years ago. This ring section is designed to provide near vision to patients. Beyond the ring section there is an aspheric surface designed to provide intermediate vision. The design is simple and has some advantages and major disadvantages, such as dependence on the aperture size to have the intermediate and near vision effect.

One attempt to improve performance of the diffraction lens design involved adding more rings and more power to the base Restor® type lens. Since diffractive effects are exploited in these lenses, they had, to some degree, the same advantages and disadvantages.

Another interesting development in diffractive lens design is the PhysIOL® diffractive lens. It is similar to the diffractive designs described above, but adds two portions that are interlaced, that is, one portion for providing intermediate vision and another portion for providing near vision. Theoretically, no matter how large a patient's pupil, both portions should be present within the pupillary space of the eye, and thus a patient should obtain acceptable near and intermediate vision, with distance vision being provided by a central portion of the lens. Both diffractive portions are apodized, so that the grooves are deeper near the center of the lens, becoming very shallow as the radial distance increases.

Another version of a multifocal lens, based on a different principle, is a lens manufactured by Oculentis®. It is similar in principle to a bifocal spectacle lens, that is, there are two curvatures present on the lens that provide for the optical performance of the lens. The lower part of the lens has added power, for example, 2.0 diopters (D), and produces a best focus for objects further away (distance vision). From an optics point of view, such a lens produces a modulation transfer function (MTF) and through-focus-response similar to the PhysIOL® lens, although there have been reports of coma and glare with this lens.

As explained above, the previous designs were created for cataract surgery only. Although the prior art designs theoretically can be implemented on a negative ICL lens, in practice, such implementation would be extremely difficult.

The multi-period design described above has a set of rings on the front surface and these rings have a depth of several hundred microns, with a sloping bottom surface (blazing). A typical intraocular contact lens (ICL) negative refractive lens is only 116 microns thick at the center and at the edge the thickness increases only to 330 microns. Therefore it would be virtually impossible to cut the rings without punching through the back surface or without seriously compromising the mechanical properties of the resulting lens. Because of the physiological constraints of the eye, the thickness of the negative lens cannot be increased, as it would no longer fit into the very tight volume where such a lens is typically is implanted in the eye.

A second problem with the diffractive rings of the multi-period design is that if they are made on the front surface of the lens they will contact the iris. There is a serious danger of chafing the iris as it scrapes against the rings as the iris opens and closes in reaction to the amount of light incident on the eye. Such chafing may result in iris pigment particles being dislodged, potentially causing serious problems of inflammation and clogging of the exit channels for the aqueous humor. On the other hand, if the rings are implanted on the back surface and they accidentally touch the crystalline lens, the insult to the crystalline lens may result in formation of a cataract within the crystalline lens.

Thirdly, diffractive surfaces are traditionally used as diffraction gratings to split light into its spectrum of colors, producing chromatic dispersion. In the case of a diffractive multifocal IOL, the chromatic dispersion becomes a serious problem and the patients have to live with this effect and somehow learn to ignore it.

Regarding the double curvature design, it is complex to manufacture and patients report observing coma effects with this lens. Such a lens also exhibits many of the problems discussed above, such as the difficulty of implementing two radii of curvature on a negative lens that is already extremely thin.

Another serious problem with the double curvature design is the occurrence of glare and haloes. These problems come from the sharp transition and abrupt change in lens power where the two surfaces meet.

What has been needed, and heretofore unavailable, is an improved multifocal lens design that can be used for both refractive and cataract surgery that is optimized to provide for improved near and visual acuity. The present invention satisfies these and other needs.

### SUMMARY OF THE INVENTION

In a general aspect, the present invention includes a free form progressive multifocal lens having an optic having an even aspheric shape. In some aspects, the even aspheric shape includes an optic having a basic conic shape on top of which an even polynomial of up to a 16^{th} order is overlaid. In such a shape, the radius of the optic varies from point to point along a radius moving out from the center of the lens.

In another aspect, the present invention includes a method for generating commands that can control a lathe to cut a free form progressive multifocal optic from a lens blank.

In yet another aspect, the present invention includes an implantable lens for improving the visual acuity of a patient, comprising: a free form progressive multifocal optic optimized to provide at least improved distance and near focus. In still another aspect, the lens includes a haptic for fixating the lens optic within an eye.

In a further aspect, the optic has a basic conic shape with an even 16^{th} order polynomial superimposed on the basic conic shape. In a still further aspect, the optic has an even aspheric shape. In an even further aspect, the even aspheric shape has a basic conic shape with an even 16^{th} order polynomial superimposed on the basic conic shape.

In still another aspect, the present invention includes a method for optimizing the geometry of a free form progressive multifocal optic, comprising: entering constants and parameters into an optimization engine; generating an optimization output; inputting the optimization output into a coordinate generator; and operating a lathe in accordance with output from the coordinate generator to cut a multifocal optic.

In yet another aspect, the constants may include, but are not limited to, object distance for distance vision, object distance for near vision, desired center thickness of the lens, desired edge thickness of the lens, desired optic diameter of the lens, and a desired posterior curvature of the optic of the lens.

In another aspect, the variables may include, but are not limited to, two or more constants to describe an aspheric surface. In another aspect, the variables may include, but are not limited to, eight constants needed to define a 16^{th} order polynomial.

In still another aspect, a merit function may be selected and used as an input for the optimization engine.

In another aspect, the optimization output may be twenty one constants that describe the optical surface and geometry of the lens. In one aspect, thirteen constants describe the aspheric optic surface and optic geometry. In another aspect, eight of the constants describe a 16^{th} order even polynomial.

In yet another aspect, the output from the generator includes point by point X and Z coordinates.

Other features and advantages of the invention will become apparent from the following detailed description, taken in conjunction with the accompanying drawings, which illustrate, by way of example, the features of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic representation showing a ray tracing through a free form progressive multifocal lens in accordance with one embodiment of the present invention.

FIG. 2 is a ray tracing of a lens having one configuration. The object is placed at infinity and a 2.5 mm aperture is placed in front of the lens. This allows the center of the lens to be optimized for distance vision.

FIG. 3 is a ray tracing of a lens having a second configuration. The object is placed at 400 mm (2.5 diopters added power) from the eye and a 2.5 mm obscuration is placed in front of the lens. This allows the periphery of the lens to be optimized for near vision.

FIG. 4 is a ray tracing of a lens having a third configuration. No aperture or obscuration in front of the lens in this configuration.

FIG. 5A is a graphical representation of FFT MTF of a lens before optimization, corresponding to the configuration of FIG. 2 (distance vision, center of the lens).

FIG. 5B is a graphical representation of FFT MTF for the lens of FIG. 5A after optimization, corresponding to the configuration of FIG. 2 (distance vision, center of the lens).

FIG. 6A is a graphical representation of FFT MTF for a lens before optimization, corresponding to the configuration of FIG. 3 (the periphery of the lens, near vision).

FIG. 6B is a graphical representation of MTF for the lens of FIG. 6A after optimization.

FIG. 7A is a graphical representation of FFT MTF for a lens before optimization, corresponding to the configuration of FIG. 4, which includes the full lens, simulating distance vision with dilated pupil, scotopic condition.

FIG. 7B is a graphical representation of MTF for the lens of FIG. 7A after optimization.

FIG. 8A is a graphical representation of FFT MTF for a lens at 50 line pairs per mm versus object position, from 0.250 m to 20 m.

FIG. 8A is a graphical representation of FFT MTF for the lens of FIG. 8A at 50 line pairs per mm versus object position.

FIG. 9A is a graphical representation of through focus response for aperture=5 mm.

FIG. 9B is a graphical representation of through focus response for aperture=4.5 mm.

FIG. 9C is a graphical representation of through focus response for aperture=4.0 mm.

FIG. 9D is a graphical representation of through focus response for aperture=3.5 mm.

FIG. 9E is a graphical representation of through focus response for aperture=3.0 mm.

FIG. 9F is a graphical representation of through focus response for aperture=2.5 mm.

FIG. 9G is a graphical representation of through focus response for aperture=2.0 mm.

FIG. 10 is a graphical representation of MTF TFR of an optimized 20.0 D free form multifocal lens.

FIG. 11 illustrates a series of image simulations for a range of apertures for near and distance vision.

FIG. 12 is a block diagram illustrating an embodiment of a method of designing a free form progressive multifocal lens.

### DETAILED DESCRIPTION OF THE PREFERRED EMBODIMENTS

The free form progressive multi-focal refractive lens in this invention is a type of intra-ocular lens that can be used in cataract and refractive surgery. Its unique features make it a good choice to provide both distance and near vision for two age groups. Cataract patients tend to be older (60+ year olds), while refractive surgery is more common in younger patients, in their 30s and 40s.

In one embodiment, the invention is a refractive-only lens, with a free form or progressive surface. This lens design is a more complex surface than the simple spherical or conic surfaces of prior art refractive type multifocal lenses, whose optical properties can be described by a single number such as radius of curvature only in the case of a spherical lens, or by two numbers, such as a radius and a conic constant for an aspheric lens surface.

In an embodiment of the present invention, the free form multifocal lens has a base conic surface, over which is laid s surface described by an even polynomial, with order up to and including the 16th order. Such surfaces are called “even asphere”, “progressive surfaces” or “free-form surfaces” to highlight the fact that if a radius of curvature measurement is attempted on this surface, the radius will be found to vary from point to point, moving radially outward from the center of the lens. However, such a lens is still symmetrical in azimuth.

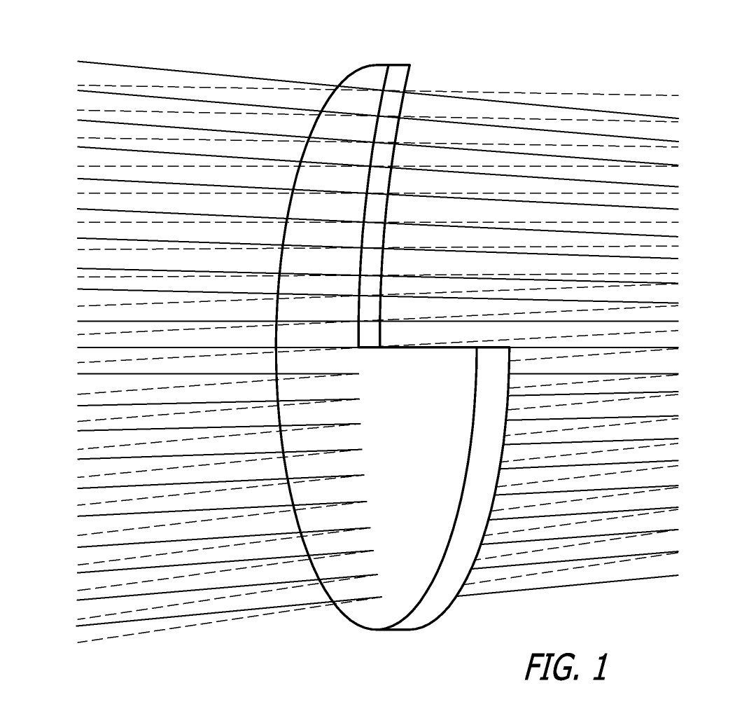

FIG. 1 is a schematic representation showing a ray tracing through a free form progressive multifocal lens 100 in accordance with one embodiment of the present invention where the front surface of the lens is an even asphere. In this embodiment, the front surface of the lens has a base conic described by a base radius of curvature and conic constant, on top of which a 16th order even polynomial is superimposed. The resulting surface is smooth and refractive-only. The lens is shown in “¾ view”, so that both the 3-D and cross-section are visible.

This type of lens cannot be designed manually or in simple computer applications such as Excel®, distributed by Microsoft Incorporated. Instead, the calculations needed to produce a lens as shown in FIG. 1 are typically performed by a ray tracing software program, such as Zemax, distributed by Radian Zemax, LLC, or Code V®, distributed by Synopsis®. The specific calculations carried out may include, for example, “global optimization” or Monte Carlo techniques.

In one embodiment, the lens is designed and optimized in 3 different configurations simultaneously. The inventors have found that such an optimization is necessary to produce the best distance vision, the best near vision and the best overall lens design for a particular lens power. For example, the software program is set up to determine the parameters of the lens using a model eye, such as the ISO11979-2 eye model, which is simulated by the software program. This process is advantageous the ISO lens model is a standard model, and is used to measure manufactured lens for quality purposes.

The desired free form lens design parameters are established by providing inputs for three different configurations, which are then simulated and optimized using methods such as the Monte Carlo technique described above. Determining the optimum lens design in each of the three configurations, and then optimizing the design over all three configurations has been found to provide an acceptable compromise that provides a lens with the best optical properties for providing near, intermediate and distance vision over a broad range of lens powers. Those skilled in the art will understand that, when reference is made a lens power, what is meant is the base optical power of the lens, which is selected by a dispenser of the lens to correct a particular visual problem.

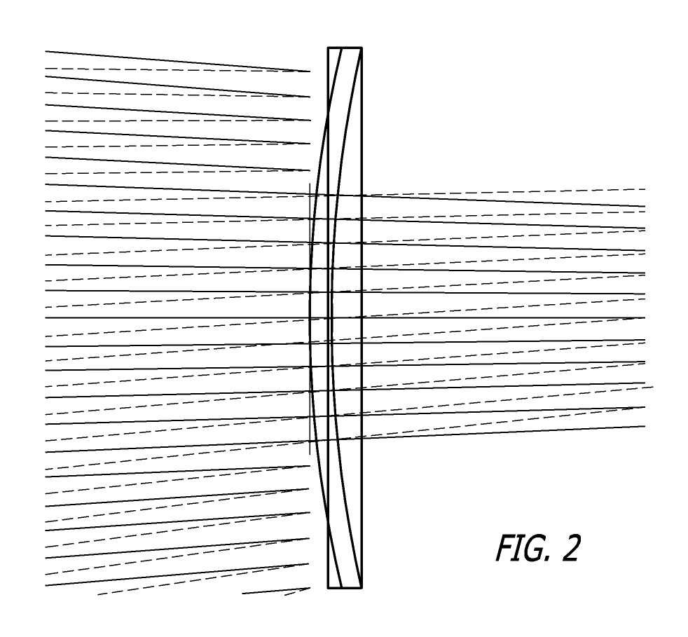

Configuration 1 of the above described optimization is illustrated in FIG. 2. FIG. 2 is a ray tracing result of a simulation that optimizes the lens for distance vision. In this simulation, the object is placed at infinity and an aperture, which simulates a particular pupillary diameter or an eye, is placed in front of the lens. In this configuration, the aperture has a circular opening 2.5 mm in diameter. Light rays launched from the object at infinity pass through the cornea of the model eye and enter the lens only through its central 2.5 mm, which allows the center of the lens, which typically provides the majority of correction for distance vision, to be optimized. This simulates the optic condition that exists for a patient in bright daylight (photopic conditions), where the pupil opening of the patient is typically small, in the order of 2 to 3 mm, and the patient needs to have clear distance vision. In this simulation, light rays launched from the object are launched both on axis, and tilted 2.5 degrees.

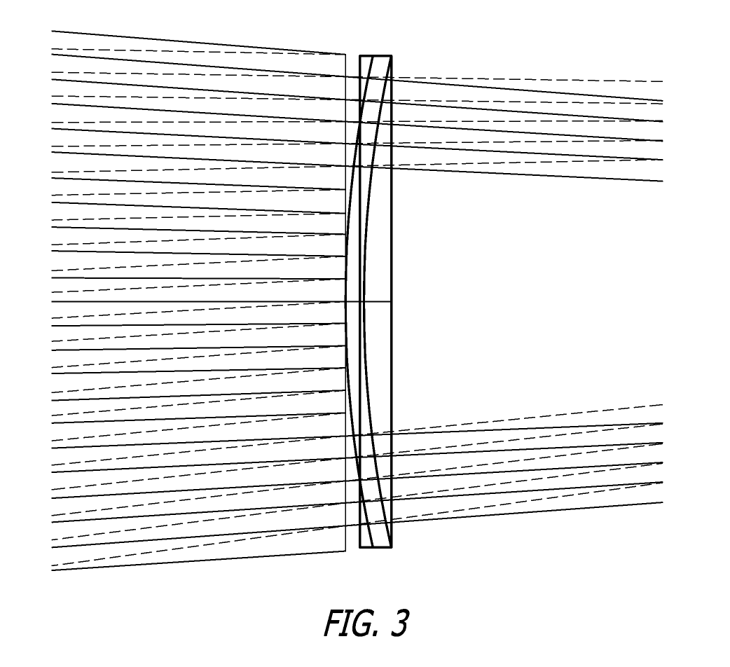

Configuration 2 of the above described optimization is illustrated in FIG. 3. In this simulation, the object is placed at 400 mm from the eye, to simulate near vision and an added power of 2.5 diopters (1000 mm/400 mm). Instead of the aperture in configuration 1, an obscuration is placed in front of the lens, with the same diameter as the aperture in configuration 1. The obscuration stops all rays that hit it, allowing only rays that impinge upon the lens beyond a central area having a diameter of 2.5 mm to proceed through the lens. This simulation optimizes the periphery of the lens.

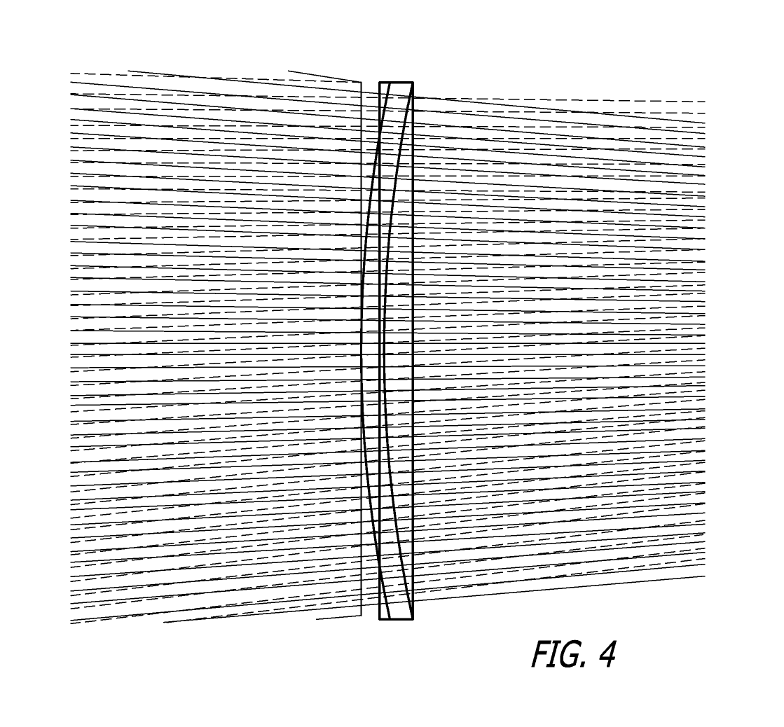

Configuration 3 of the above described optimization is illustrated in FIG. 4: in this simulation, the light is launched from infinity, as in configuration 1, but there is no aperture or obscuration in front of the lens. As shown in FIG. 4, the entire surface of the lens is illuminated by the light rays. All optical performance functions, such as, for example MTF, spot size and through-focus response, are calculated for this configuration as well.

In all of the configurations described above, light is launched at the lens with both zero angle of incidence (the angle between the light rays and the normal to the lens) and also tilted at 2.5 degrees, which would reach the retina at the edge of the fovea.

The lens optimization process will now be described. In a typical embodiment of a minus power lens, such as, for example, a lens with a base power of −3.00 D, also known as a negative lens, that is normally used in refractive surgery to correct myopia. Those skilled in the art will appreciate that the same optimization process may be carried out on a positive power lens, such as, for example, a lens with a base power of +12.00 D, such as might be used to correct a patient's vision after cataract removal.

The front surface of the negative power lens may be designed to have a free-form even asphere surface that is subject to optimization. All geometrical parameters of this surface, for example, the radius of curvature of the base surface and its conic constant, plus the 8 terms of the 16th order even polynomial (10 parameters in total) are turned into variables and the software is allowed to change them to produce a better lens in both configurations 1 (distance) and 2 (near vision) simultaneously. The simulation described above with reference to configuration 3 does not take part in the optimization and it is performed to provide for checking the final result of the optimizations of configurations 1 and 2, and ultimately, the finished optimized lens.

The only other variable subject to optimization is the distance of the image surface. Therefore, the back surface of the lens and also the lens center thickness are held constant. IN this manner, a total of 11 geometrical parameters can be changed by the software to optimize the lens.

The knowledge of what constitutes a good lens is coded as a merit function containing tens of lines of arguments. Each argument line represents an optical property of the lens, such as, for example, MTF, optical path difference, and the like, and a high target value for the merit function is given. The software calculates the present value of these parameters and subtracts it from the target value. An RMS (Root Mean Squared) value that is the sum of all differences between the present value and the target value, multiplied by individual weights assigned to various variables and parameters is calculated and this is the present value of the merit function. The software program optimizes the simulation by attempting to minimize this value by making changes to the 11 variables of the lens and continuously running simulations in a Monte Carlo fashion until the value is minimized by some combination of the 11 variables. In general the optimization process requires several million changes to the lens, essentially trying several million different lens designs to find the designs with the lowest merit function. In most cases, running 10 million cases is sufficient to produce a reasonably optimized lens.

The process described here can be used to optimize both negative and positive lenses. An example of the optimization process is presented, applied to the design of a 20.0 D cataract lens. In this example, the commercially available Zemax software was used to design the lenses, with special modifications. Each surface of the lens is defined by a rotationally symmetric polynomial aspheric surface, which is described by a polynomial expansion of the deviation from a spherical or aspheric surface. The even asphere surface uses only the even powers of the radial coordinate to describe the asphericity, leading to rotational symmetry.

In alternative embodiments, a more general surface containing a cylinder component could be designed as well, in which case both odd and even terms of the polynomial could be used. In still another embodiment, an extended asphere, containing up to 480 polynomial terms could also be used to design these lenses.

In this example, each surface of the lens may be described in general terms by the following equation:

$$z = \frac{{cr}^{2}}{1 + \sqrt{1 - ( 1 + k ) \cdot {c}^{2} {r}^{2}}} + {α}_{1} {r}^{2} + {α}_{2} {r}^{4} + {α}_{3} {r}^{6} + {α}_{4} {r}^{8} + {α}_{5} {r}^{10} + {α}_{6} {r}^{12} + {α}_{7} {r}^{14} + {α}_{8} {r}^{16}$$

The terms in this equation have the following meanings:

z=surface sag

c=1/R is the surface curvature, where R is the surface radius of curvature.

r²=x²+y² is the square of the surface radial coordinate.

k is the conic constant, which is less than −1 for hyperbolas, −1 for parabolas, between −1 and 0 for ellipses 0 for spheres and greater than zero for ellipsoids.

α₁ to α₈ are the even asphere coefficients and are used to superimpose the polynomial on the aspheric surface. Note that if all alphas are zero the equation above describes a standard asphere and if k=0 as well, the equation reduces to a standard spherical surface.

The radius of curvature (R), the conic constant (k) and the 8 alpha parameters are set as variables in the Zemax software program, giving a total of 10 variables per surface or 20 variables for the back and front surfaces of the lens. The lens center thickness can be set as a variable as well, increasing the total number of potential variables to 21.

In addition, several configurations may be set up in the Zemax software program, as described above, where the distance between the light source and the lens inserted in the model eye is varied, as well as other parameters, such as the pupil diameter of a model eye. The Liou and Brennan model eye or the ISO model eye, or any other suitable model eye, can be used in setting up the simulation to perform the optimization process. The ISO model eye is used in this example.

In the following example illustrated in Table 1, four configurations are defined. Line 2 of the table shows that the distance from the lens to the light source (the source of rays to be traced) varies from 500 mm to 1E10 mm (=1E7 meters or 10,000 km, essentially infinity). Line 3 of the table sets the distance from the last surface in the model eye to the image plane as a variable in configuration 1 and the other configurations “pick-up” the same value, so that this distance is the same in all configurations. Line 4 of the table is the semi-diameter of the pupil, showing that Configs 1 and 3 are set for scotopic viewing conditions, with the pupil diameter open to 5 mm (2×2.5 mm), while Configs 2 and 4 are set for photopic viewing conditions, with a pupil diameter of 3 mm (2×1.5 mm).

TABLE 1

| Active: ¼ | Config 1 | | Config 2 | | Config 3 | | Config 4 | | |
|---|---|---|---|---|---|---|---|---|---|
| 1: MCOM | 0 | Near 2.5 | | Near 1.5 | | Medium 2.5 | | Infinity 1.5 | |
| 2: THIC | 0 | 500.0000000 | | 500.0000000 | | 700.0000000 | | 1.0000E+010 | |
| 3: THIC | 10 | 3.898759897 | V | 3.898759897 | P | 3.898759897 | P | 3.898759897 | P |
| 4: SDIA | 6 | 2.500000000 | | 1.500000000 | | 2.500000000 | | 1.500000000 | |

The 21 parameters described above are set as variables and a merit function is constructed using the Zemax software program to instruct the ray tracing software on how to optimize the performance of the lens.

Many parameters can be used to describe what constitutes a well performing lens, that is, a lens that provides for the best combination of distance and near vision, are included in the merit function. In this example, substantial weight is given to MTF parameters. Other parameters may be used as well, such as Strehl ratio, encircled energy, wavefront error, and the like. Table 2 below illustrates an example of a merit function and each line is described in detail below.

TABLE 2

| Oper # | Type | Samp | Wave | Field | Freq | Grid | | | Target | Weight | Value | % Contrib |
|---|---|---|---|---|---|---|---|---|---|---|---|---|
| 6: Zern | Zern | 11 | 1 | 2 | 1 | 1 | 0.00 | 0 | 0.00 | 0.00 | −1.03909 | 0.00000000 |
| 7: EFLX | EFLX | 7 | 8 | | | | | | 50.00 | 1000.0 | 50.01574 | 0.01478166 |
| 8: EFLY | EFLY | 7 | 8 | | | | | | 50.00 | 1000.0 | 50.01574 | 0.01478166 |
| 9: MTFA | MTFA | 3 | 0 | 1 | 50.00 | 1 | | | 0.90 | 500.00 | 0.431364 | 6.55481287 |
| 10: BLNK | BLNK | | | | | | | | | | | |
| 11: CONF | CONF | 2 | | | | | | | | | | |
| 12: MTFA | MTFA | 3 | 0 | 1 | 50.00 | 0 | | | 0.90 | 1000.0 | 0.221388 | 27.4891249 |
| 13: BLNK | BLNK | | | | | | | | | | | |
| 14: CONF | CONF | 3 | | | | | | | | | | |
| 15: MTFA | MTFA | 3 | 0 | 1 | 50.00 | 0 | | | 0.90 | 500.00 | 0.190169 | 15.0382714 |
| 16: BLNK | BLNK | Lens Mechanical Properties | | | | | | | | | | |
| 17: CONF | CONF | 4 | | | | | | | | | | |
| 18: MTFA | MTFA | 3 | 0 | 1 | 50.00 | 1 | | | 0.90 | 1000.0 | 8.3e−003 | 47.4683494 |
| 19: ETGT | ETGT | 7 | 0 | | | | | | 0.30 | 1000.0 | 0.300000 | 0.0000000 |
| 20: ETLT | ETLT | 7 | 0 | | | | | | 0.40 | 1000.0 | 0.40000 | 0.0000000 |
| 21: ETVA | ETVA | 7 | 0 | | | | | | 0.00 | 0.00 | .372071 | 0.0000000 |
| 22: DMFS | DMFS | | | | | | | | | | | |
| 23: BLNK | BLNK | Sequential merit function: RMS wavefront centroid GQ 3 rings 6 arms | | | | | | | | | | |
| 24: CONF | CONF | 1 | | | | | | | | | | |
| 25: BLNK | BLNK | No default air thickness boundary constraints | | | | | | | | | | |
| 26: BLNK | BLNK | No default glass thickness boundary constraints | | | | | | | | | | |
| 27: BLNK | BLNK | Operands for field 1 | | | | | | | | | | |
| 28: OPDX | OPDX | | 1 | 0.00 | 0.00 | 0.3357 | 0.00 | | 0.00 | 0.8727 | 1.728486 | 0.15563133 |
| 29: OPDX | OPDX | | 1 | 0.00 | 0.00 | 0.7071 | 0.00 | | 0.00 | 1.3963 | 1.650951 | 0.22717162 |
| 30: OPDX | OPDX | | 1 | 0.00 | 0.00 | 0.9420 | 0.00 | | 0.00 | 0.8727 | −4.37001 | 0.99478665 |
| 31: CONF | CONF | 2 | | | | | | | | | | |

The following is a description of headings of each column in the above table:
Oper #: operator number in the merit function and its 4 character name. These are the operators whose values describe how well the lens will perform.
Type: type of operator is identical to its 4 character name in the example.
Samp[ling]: used by some operators, such as MTFA, to describe how many rays are sampled at the pupil.
Wave[length]: the light wavelength
Field: 1 means the light is incident at zero degrees to the surface normal.
Freq[uency]: the spatial frequency where MTFA is calculated. In the present example, 50 line pairs per millimeter was used, although other values may be used.
Grid: This is a Zemax software program internal parameter controlling how the software program performs calculations.
Target: This is the target value for each particular operator that Zemax is instructed to determine. For example, EFLX and EFLY are set at a target of 50 mm. This means the lens in this example is a 20 diopter lens (1000 mm/50 mm = 20D)
Weight: This is the relative importance of this parameter. For example, the weights for EFLX, EFLY are set at 1000 and contribute more. Other parameters have lower weights, indicating that they are not as important to the optimized lens.
Value: This column gives the present value for each operator, given the present values of the 21 variables. For example, EFLX value is 50.01574 and contributes with only 0.01478166% of the total merit function.
% Contrib: The contribution of each operator to the merit function is given in the last column. Ideally, the value listed in the “Value” column should to be as close as possible to the value in the “Target” column, so that the contribution is reduced. The Zemax software program will choose values for the 21 variables that minimize the sum squared of the contributions of all operators.

Internally, the Zemax software program constructs a mathematical description of the merit function from the above described operators, illustrated by the equation below:

$${MF}^{2} = \frac{{\sum }^{ } {{W}_{i} ( {V}_{i} - {T}_{i} )}^{2}}{{\sum }^{ } {W}_{i}} ,$$

Where Wᵢ is the weight of operand “i”, Vᵢ is the operand current value, Tᵢ is the target value and the subscript “i” indicates the operand number, that is, its row number in the merit function spreadsheet. The sum index “i” runs over all operands in the merit function. Clearly, if the weight Wᵢ is set to zero for a particular operand, it has no effect on the value of the merit function.

The lines in the merit function illustrated in Table 2 above will now be described:

Line 6: The Zernike 11th coefficient describing spherical aberration is included, but its weight is zero, which means it is here for information only, so that the Zemax software program reports its value as the lens is optimized, but it is not used in the optimization process directly, and thus is not needed for the optimization of a lens design.

Lines 7 and 8: EFLX and EFLY: Effective focal lengths in the X and Y directions. EFFL, which is an average of both EFLX and EFLY could be used as well. This allows the Zemax software program to design the lens with the correct power.

Line 9: MTFA: Average MTF for all azimuthal angles. This parameter is set at a frequency of 50 line pairs per mm, with a high target value. Other frequencies may be used as well as other values for the weight. In this example, the weight for this MTFA is set to 500 and it is part of configuration 1. Similar MTF operators such as MTFS and MTFT may be used as well.

Line 11: CONF2: The lines below this line in Table 2 describe operators for the second configuration, until a new CONF parameter is found. In this example, MTFA in line 12 with a weight of 1000, before a blank line is found and so the function jumps to CONF3.

After the MTFA for all 4 configurations are set with their target values and weights, values for the lens edge thickness are set. This is controlled by the operators below:

Line 19: ETGT: Edge Thickness Greater Than. This parameter forces the Zemax software program to control the lens thickness such that the resulting lens is not too thin.

Line 20: ETLT: Edge Thickness Less Than: This parameter forces the Zemax software program to produce a lens that is not too thick.

Line 21: ETVA: Edge Thickness Value: In this example, the Zemax software program did not report the edge thickness in lines 19 and 20, because the program produced a lens satisfying these constraints. Therefore ETVA is here only to tell the user the current Edge Thickness value. Notice that its weight is zero, and as such, does not take part in the optimization.

Line 22 to Line 31: These lines use the standard “Default Merit Function” in the Zemax software program and allow the program to minimize Optical Path Difference error. This is a standard technique used in ray tracing and these default merit function operators are added to the operators described above.

Armed with this merit function and the 21 variables set previously, the ray tracing Zemax software program uses its own proprietary algorithms to make changes to the 21 variables and calculate the merit function MF2 given above. The program can be set to continue making changes to the variables and testing the new values of MF2 until the lens designer stops it or it can be set to stop automatically once the changes in the variables no longer produce changes in MF2 larger than a very small, internally controlled number.

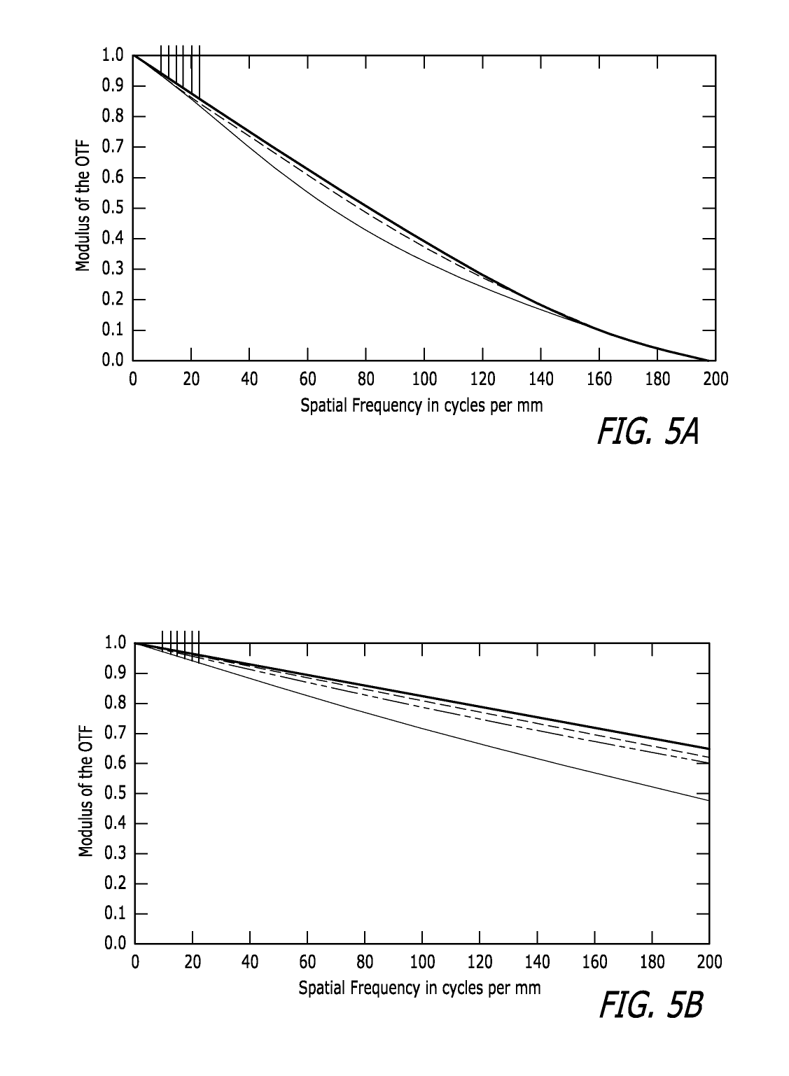

Referring now to FIG. 5A, the MTF for configuration 1 (2.5 mm aperture, thus allowing only the center of the lens to be illuminated, and optimizing for distance vision) at the beginning of the optimization process is shown. The lens MTF is shown in blue and is overlapping the diffraction limit curve shown in black, that is, the lens is diffraction limited for distance vision at this small aperture.

FIG. 5B) shows the MTF after running 10 million cases through the optimizing process. The top curve in black is the diffraction limit, blue curve is the MTF for light on-axis and the two green curves are the sagittal and tangential MTFs for light at 2.5 degrees. The lens MTF after optimization is still almost diffraction-limited for photopic conditions (small pupil, light going through the center of the lens only).

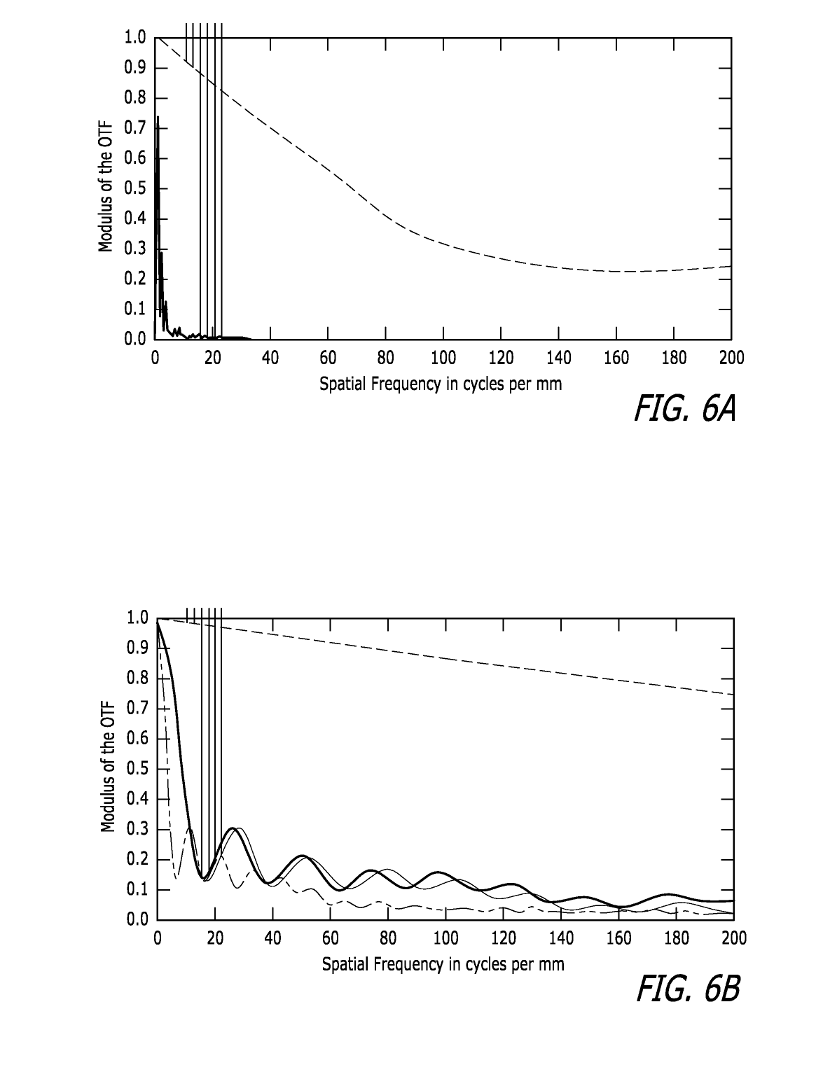

FIG. 6A shows the MTF for configuration 2 (light impinging on the periphery of the lens only, optimized for near vision) at the beginning of the optimization process. The performance for near vision is extremely poor. The low diffraction limit is an artifact caused by the inclusion of the obscuration in front of the lens.

FIG. 6B shows the MTF after running 10 million cases through the optimizing process. Again, the top curve in black is the diffraction limit, blue curve is the MTF for light on-axis and the two green curves are the sagittal and tangential MTFs for light at 2.5 degrees. Although the MTF is much lower than the distance vision case, it is still above 0.2 at 50 line pairs/mm. The MTF in this example is extremely poor initially, but shows good improvement for near vision after optimization.

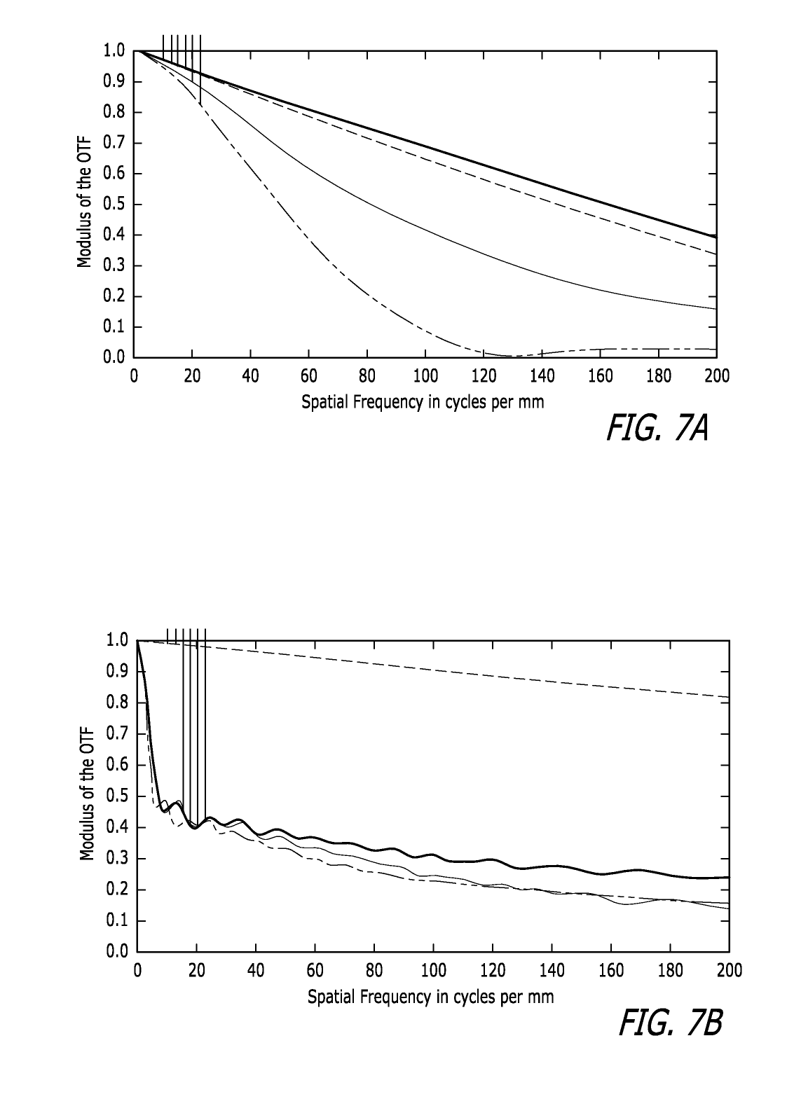

FIG. 7A shows the MTF for configuration 3 (light impinging on the entire lens, simulating distance vision with a dilated pupil, scotopic\condition) at the beginning of the optimization process.

FIG. 7B shows the MTF of the lens after running 10 million cases through the optimizing process. Although the MTF of resultant lens is degraded compared to the lens of FIG. 5B, it is still a reasonable value of 0.38 at 50 line pairs per millimeter. The human eye, for comparison is 0.1 at the same spatial frequency.

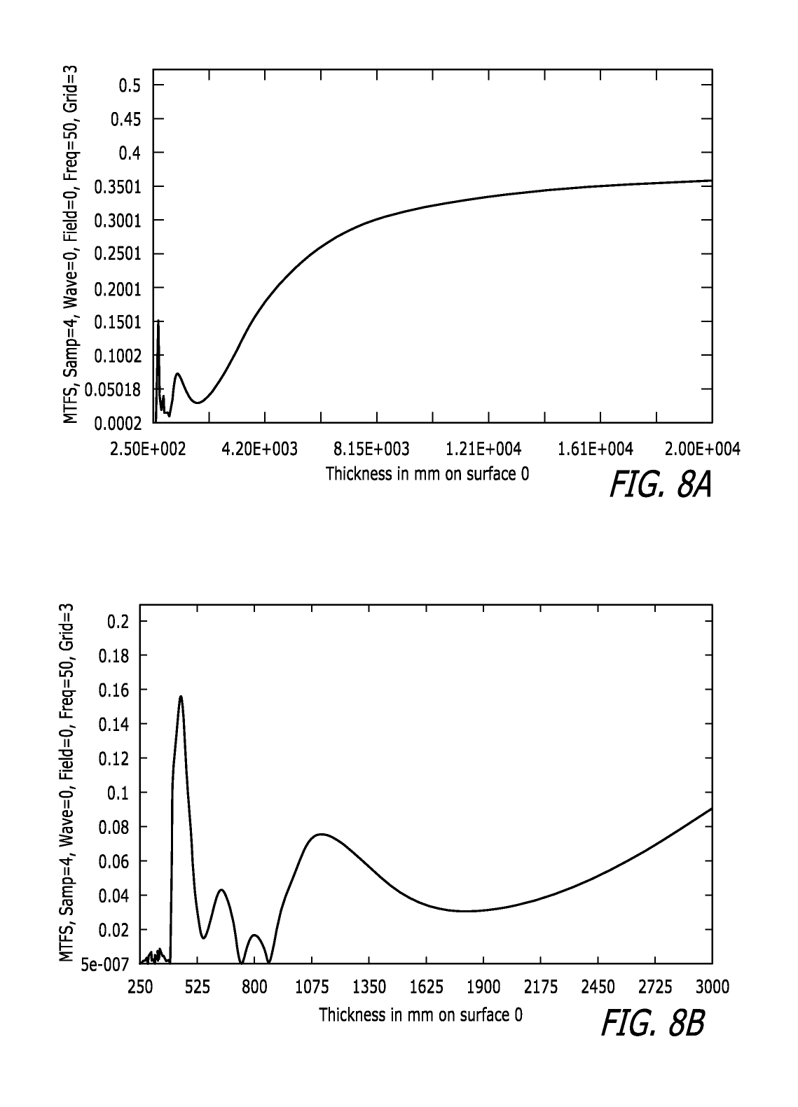

FIGS. 8A-B show the FFT MTF for configuration 3 (full lens) as a function of object position, from 250 mm to 20 meter. Notice that for distance vision (above about 12 meters), the MTF is almost constant at about 0.35 (FIG. 8A). FIG. 8B, which shows the MTF for object ranges from 250 mm to 3 meters illustrates that for near vision, this lens produces an MTF value of 0.16 at 400 mm.

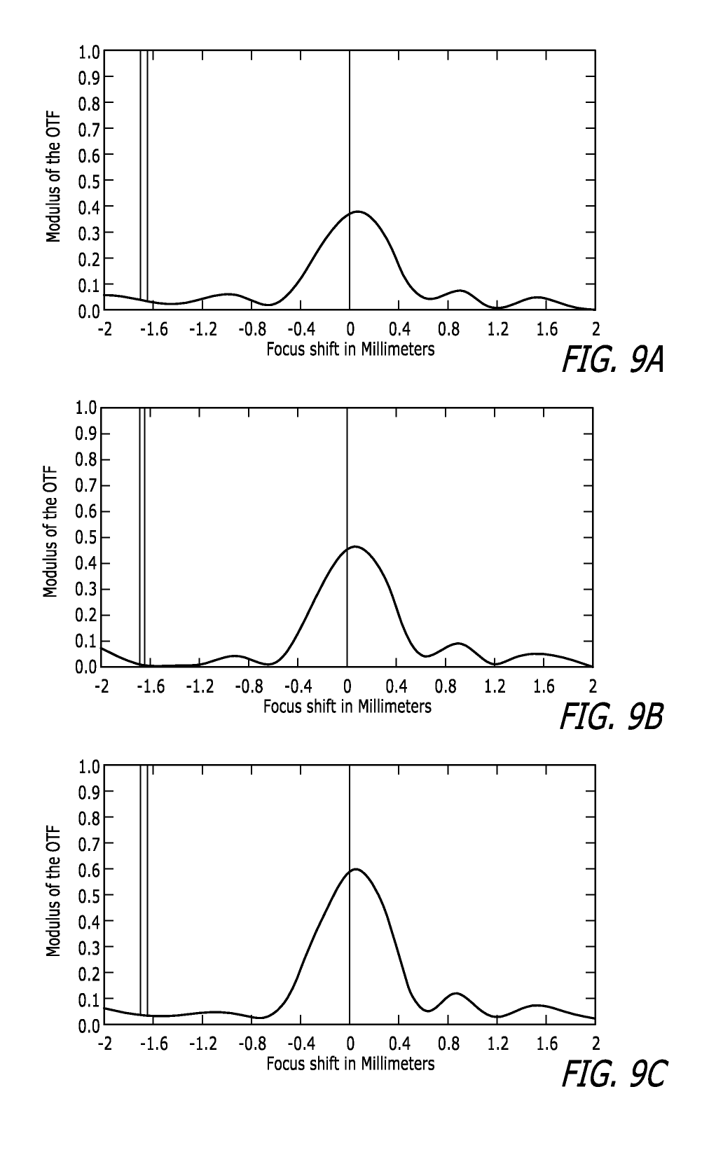

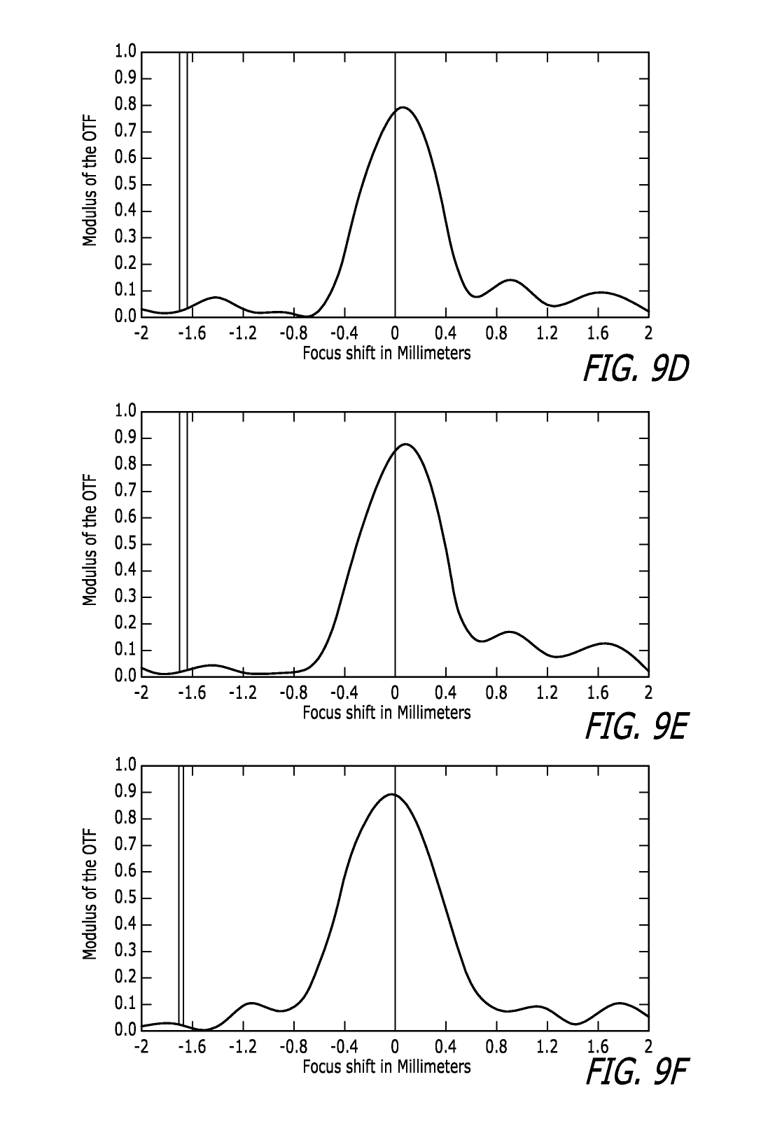

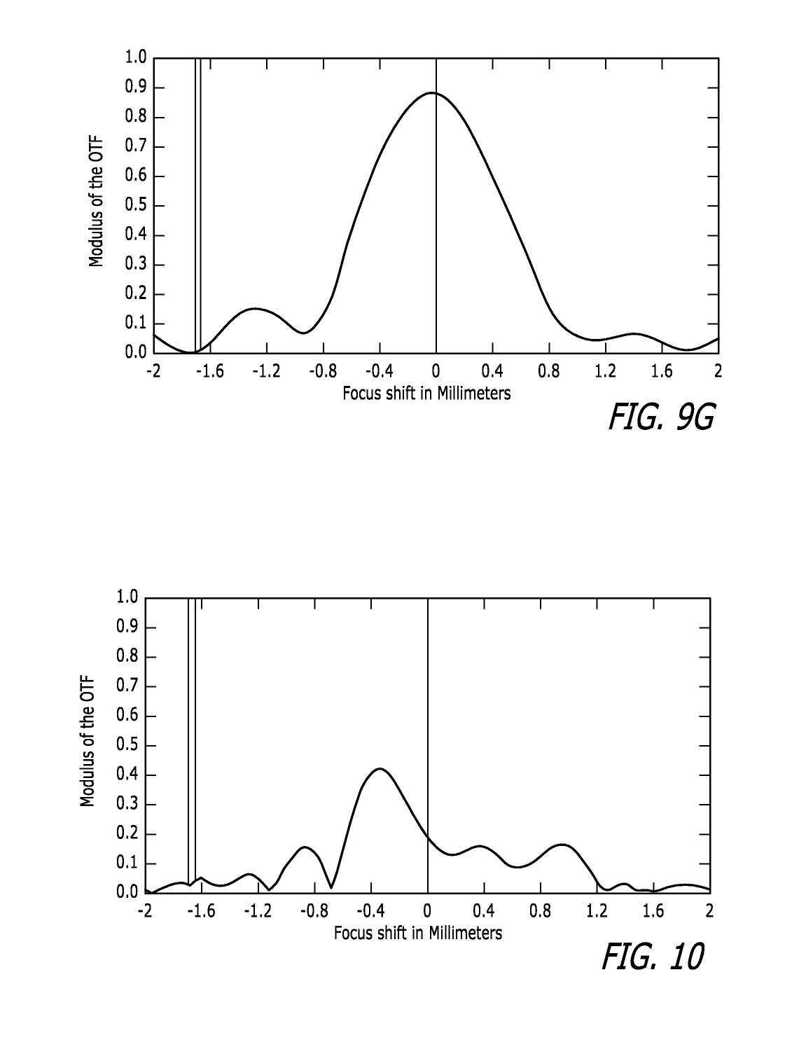

FIGS. 9A-G show how the MTF “Through-Focus-Response” (TFR) changes with image position for configuration 3 (full lens). FIG. 9A shows the TFR x focus shift for a full aperture of 5 mm and in the other figures the aperture is reduced in steps of 0.5 mm until it reaches 2 mm only in FIG. 9G. These figures show that the TFR peak width remains essentially the same as the aperture is reduced, indicating that the added multifocal power does not depend strongly on the aperture. As expected, the MTF TFR peak height increases as the aperture decreases, indicating a smaller contribution from aberrations.

The following is an example of an optimization that was carried out to design a lens with a base power of 20 D using the processes described with reference to Tables 1 and 2 above. In this example, the following parameter were determined:

Front Radius=RF=14.69189762 mm,

Front conic=kF=33.77664176,

Back Radius=RB=−14.69189762 mm,

Back Conic=KB=33.77664176

Center thickness=tc=1.217 mm,

Edge Thickness=0.372 mm, and

Diameter=5.0 mm.

In this example, one constraint on the optimization was to produce a lens that is symmetrical, so that the front surface is identical to the back surface. Such a construction provides advantages for manufacturing and for the doctor implanting the lens during surgery. For example, during manufacturing of the lens, operators do not need to remember which side of the lens they are working on. For the surgeon and the patient, there is no danger of implanting the lens backwards, as the sides are identical. While such a lens is advantageous, other lens designs where the front and back surfaces are different may be required in certain situations. These designs may also be optimized in accordance with the various embodiments of the present invention.

Below are the alpha coefficients that were generated during optimization of the exemplary 20 D lens above:

α_{1F}=−1.746918749E−3;

α_{2F}=1.2891541066E−3;

α_{3F}=−2.394731319E−4;

α_{4F}=−7.395684842E−6;

α_{5F}=−5.428966416E−5;

α_{6F}=1.309282366E−5;

α_{7F}=−7.609584642E−7;

α_{8F}=−4.857728161E−8.

For the coefficients for the back surface, the coefficients of the back surface (α_{1B}-α_{iB}) have equal values to the corresponding coefficients for the front surface, but with opposite signs. This makes the front and back even asphere polynomial surfaces identical for this exemplary lens.

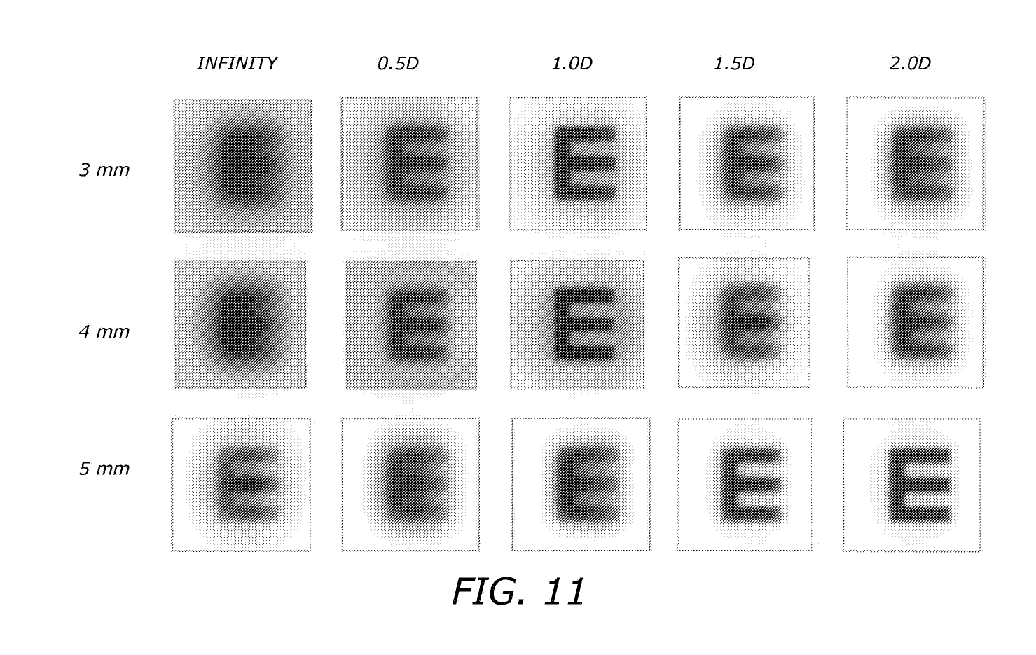

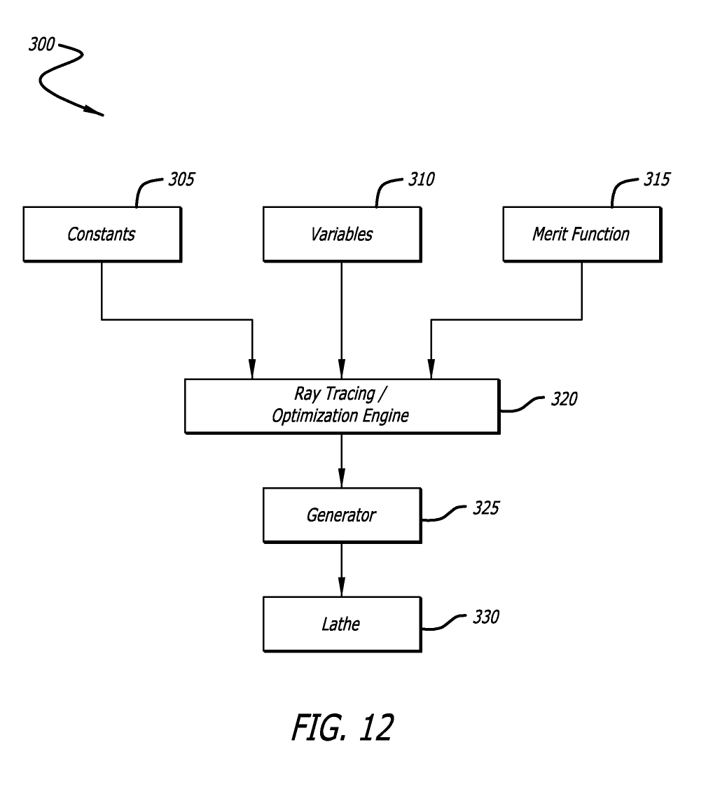

The resulting lens quality can be evaluated using Modulation Transfer Function “Through-Focus” Response (MTF TFR). FIG. 10 is plot of modulus of the optical transfer function (OTF) as a function of MTF TFR for the exemplary 20 D lens optimized above. The plot shows that this lens has a high MTF for a wide range of focus shifts, which translates into good quality vision from near to distance vision, as shown by the simulations of the letter “E” in FIG. 11. These simulations show acceptable image quality in the first column when the light source (the E) is at infinity, for a pupil diameter of 3, 4 and 5 mm. In the second column the light source is at 2 meters in front of the eye (1 meter/2 meters=0.5 D, as indicated in the column heading). The image quality is better for all pupil diameters in this case. The 3rd column shows image quality for all pupil apertures when the light source is at 1 meter in front of the lens and the 4th column is for the case when the light source is at 666 mm in front of the lens. Finally the last, 5th column, shows the image quality when the light source is 500 mm in front of the lens, for all pupil diameters. The image quality for this particular lens is best in this last situation, that is, for near vision. It is also possible to optimize the lens to produce best image quality for distance vision.

As stated previously, the Lens design optimization in accordance with the present invention is useful for designing lenses to be used in both cataract and refractive surgery. In contrast, the multifocal designs presently available can be used to replace cataract lenses only, and would be a poor choice if implemented as a refractive-surgery lens to correct myopia.

The free-form progressive multifocal (FFPM) surface of lens produced in accordance with the various embodiments of the present invention are smooth, in contrast to the rough surfaces of typical diffractive style intraocular lenses. This is particularly advantageous, as the iris slides over the front surface of an ICL (refractive surgery lens), which is typically implanted in an eye with an intact crystalline lens. The smooth FFPM surface will not chafe the iris if designed as the front surface, or the crystalline lens, if it is implemented as the back surface. Moreover, the smooth free-form progressive multifocal surface does not create haloes and glare or other Weber's Law optical aberrations which could result in visual problems for a patient.

FFPM lens designs preserve the physiological shape of ICL lenses currently available, while providing multifocal vision. There is very little room and very severe physiological constraints on any lens to be implanted in the sulcus or on top of the zonules of the human eye. If the implanted lens touches the crystalline lens, it can cause a cataract. On the other hand if it makes the iris vault too much, this can cause angle closure and lead to increased ocular pressure and glaucoma.

The FFPM design described above is easier to manufacture than a diffractive lens. There is no need to control the spacing, depth and “blazing” angle and apodization factor of a complex diffractive optical element as for the PhysIOL® lens. Further, a toric surface can also be added to this design by changing the base conic surface to which the 16th order polynomial is added.

The free-form progressive multi-focal surface lens may also be designed with more than the two present configurations for distance and near vision. For example, it could be designed for distance, intermediate and near vision or some other similar combination. For example, lenses optimized for distance and intermediate vision only, or for intermediate and near vision only, may be designed and manufactured. The lens may also be designed with other sizes for the aperture and obscuration, to give more emphasis to near vision or distance vision.

Alternatively, the FFPM lens may be re-designed without the use of the aperture and obscuration configurations described above. For example, a single configuration might be used and conditions for distance and near or distance and intermediate vision imposed inside the merit function only.

FIG. 10 is a schematic diagram showing a method 300 constituting one embodiment of the present invention for designing an optimized FFPM lens and for generating specific commands and coordinates that may then be provided to a lathe to manufacture the FFPM lens. The method begins by entering constants 305, variables 310 and a selected merit function or functions 315 as inputs to a ray tracing/optimization engine 320. Constants 305 may include, for example, but are not limited to, object distance for distance vision, object distance for near vision, desired center thickness of the lens, desired edge thickness for the lens, and a desired posterior curvature of the optic of the lens. Variables 310 may include, for example, but are not limited to, constants used to describe the aspheric surface and constants for the 16^{th} order even polynomial. For example, two constants may be needed to describe the aspheric surface and eight constants may be required for the 16^{th} order even polynomial. Merit Function 315 is typically a complex merit function that is used to allow the ray tracing/optimization program running on the engine 320 to determine which result of any given ray tracing is better or worse than any other. The results depend on the merit function or functions specified.

The output of engine 320 is typically twenty one constants that describe the optical surface and geometry of the optimized FFPM lens. Thirteen of the constants describe the aspheric optic surface and the optic geometry. Eight of the constants are used to define the 16^{th} order polynomial. These outputs, along with other constants specifying the shape of the haptics of the desired lens and other geometrical properties of the lens are input into generator 325.

Generator 325 uses appropriate software running on a computer to generate point by point X and Z coordinates that are used by lathe 330 to cut the desired shape and geometry from a lens blank to form a finished optimized FFPM lens.

Generator 325 includes software that is generally available to translate the design parameters determined by engine 321 into CNC code that can be transferred to the lathes that are used for manufacturing the FFPM lenses. In one embodiment, the generator is a proprietary programming script including appropriate commands to control a processor to carry out the functions of the generator. One or more devices such as a memory, input, output, display and printer, and communication ports may also be provided that interact with programming script running on the processor to communicate the outputted CNC code to the lathes. Alternatively, the CNC code may be provided to the lathes using a portable memory device, such as a disk, solid state memory device, or the like.

The programming script has the ability to calculate CNC code describing intraocular lenses based on optical information pertaining to the lenses optical properties on one hand and geometrical information related to the haptics of the lenses on the other hand. The programming script relies on the optics information calculated using the commercially available optical design Zemax software program as described above, because generator 325 itself does not perform any optical calculation, nor does it modify the optical design input provided by the Zemax software program.

The optical parameters describing the optical properties of the intraocular lenses are exported from engine 320 and are provided to generator 325 as an input using a text file. The generator translates this optic information into numerical coordinates (CNC code), merging them with the geometrical information relating to the haptics of the intraocular lenses and making sure a geometrically smooth transition connects the two lens regions.

It will be understood that the processes described above are incorporated into software that, when running on a computer having a processor, inputs devices, output devices, communication ports, and memory, to control the computer to carry out the processes described. The computer may be a general purpose computer that is programmed using appropriate software that is provided to carry out a specific task. Alternatively, the computer may be specifically designed to carry out only the task described. Moreover, the programs described may be incorporated into custom or, in some cases, commercially available software, or a combination of both.

While several particular forms of the invention have been illustrated and described, it will be apparent that various modifications can be made without departing from the spirit and scope of the invention.

We claim:

1. An implantable lens for improving the visual acuity of a patient, comprising:
a free form progressive multifocal optic optimized to provide at least improved distance and near focus.

2. The lens of claim 1, further comprising a haptic for fixating the lens optic within an eye.

3. The lens of claim 1, wherein the optic has a basic conic shape with an even 16^{th} order polynomial superimposed on the basic conic shape.

4. The lens of claim 1, wherein the optic has an even aspheric shape.

5. The lens of claim 4, wherein the even aspheric shape has a basic conic shape with an even 16^{th} order polynomial superimposed on the basic conic shape.

6. A method for optimizing the geometry of a free form progressive multifocal optic, comprising:
entering constants and parameters into an optimization engine;
generating an optimization output;
inputting the optimization output into a coordinate generator;
operating a lathe in accordance with output from the coordinate generator to cut a multifocal optic.

7. The method of claim 7, wherein the constants include object distance for distance vision, object distance for near vision, desired center thickness of the lens, desired edge thickness of the lens, desired optic diameter of the lens, and a desired posterior curvature of the optic of the lens.

8. The method of claim 7, wherein the variables include two or more constants to describe an aspheric surface.

9. The method of claim 7, wherein the variables include eight constants needed to define a 16th order polynomial.

10. The method of claim 7, wherein a merit function is an input for the optimization engine.

11. The method of claim 7, wherein the optimization output is twenty one constants that describe the optical surface and geometry of the lens.

12. The method of claim 11, wherein thirteen constants describe the aspheric optic surface and optic geometry.

13. The method of claim 11, wherein eight of the constants describe a 16th order even polynomial.

14. The method of claim 7, wherein the output from the generator includes point by point X and Z coordinates.
